(19) United States
(12) Patent Application Publication
(10) Pub. No.: US 2026/0094291 A1
(43) Pub. Date: Apr. 2, 2026

(54) SYSTEMS AND METHODS FOR RECONSTRUCTING 3D OBJECTS

(71) Applicants: University of Washington, Seattle, WA (US); Children's Mercy Hospital, Kansas City, MO (US); Seattle Children’s Hospital d/b/a Seattle Children’s Research Institute, Seattle, WA (US)

(72) Inventors: Michael Barbour, Seattle, WA (US); Alberto Aliseda, Seattle, WA (US); Shaunak Amin, Seattle, WA (US); John Dahl, Seattle, WA (US); Randall Bly, Seattle, WA (US); Seth Friedman, Seattle, WA (US)

(21) Appl. No.: 19/340,749

(22) Filed: Sep. 25, 2025

Related U.S. Application Data
(60) Provisional application No. 63/700,132, filed on Sep. 27, 2024.

(51) Int. Cl. G06T 7/60; A61B 1/04; A61B 1/267; G06T 5/90; G06T 7/00; G06T 7/70; G06T 7/80; G06T 17/00; G06T 19/20

(52) U.S. Cl. CPC G06T 7/60; A61B 1/04; A61B 1/267; G06T 5/90; G06T 7/0012; G06T 7/70; G06T 7/80; G06T 17/00; G06T 19/20; G06T 2207/30208

(57) ABSTRACT

Systems and methods are disclosed herein for using structure from motion (SfM) techniques to reconstruct three-dimensional (3D) surface models of tubular patient anatomies, such as the larynx and trachea, from clinical endoscopy videos. The disclosed methods may improve understanding of upper airway disease including vocal fold paralysis, laryngeal cancer, subglottic hemangiomas, subglottic stenosis, tracheal stenosis, tracheal cartilaginous sleeves, complete tracheal rings and tracheomalacia, and allow for quantitative analysis of complex laryngotracheal geometries. Quantitative measures of airway caliber and shape, which are critical for diagnostic purposes, may be obtained using the disclosed methods as a cost-effective and radiation-free alternative to relying on imaging studies. Results have demonstrated excellent resolution of reconstructions, when compared to high-resolution computed tomography (CT) scans (surface errors <0.300 mm).

### CROSS REFERENCE TO RELATED APPLICATIONS

The present application claims priority to U.S. Provisional Application No. 63/700,132 titled “SYSTEMS AND METHODS FOR RECONSTRUCTING 3D OBJECTS”, and filed on Sep. 27, 2024. The entire contents of the above-listed application are hereby incorporated by reference for all purposes.

### STATEMENT OF FEDERALLY SPONSORED RESEARCH AND DEVELOPMENT

This invention was made with government support under Grant Nos. T32DC000018 and 1R21HL172011-01A1 awarded by the National Institutes of Health. The government has certain rights in the invention.

### TECHNICAL FIELD

Embodiments of the subject matter disclosed herein relate to reconstructing a 3D surface of a patient anatomy.

### BACKGROUND

A 3D surface representation of larygotracheal patient anatomy and quantitative metrics derived from a laryngotracheal surface of a patient may be desired for patient diagnosis, treatment planning, and surgery planning. These surfaces and metrics are currently generated using CT imaging, which is expensive and exposes patients to radiation. Endoscopy is currently the gold standard technique for characterizing pediatric airway diseases, however this method is limited for quantitative analysis due to lack of three-dimensional (3D) vision, depth perception, and ability to measure airway dimensions. As a result, to perform the surface generation and quantitative analysis, one or more cross-sectional imaging studies (eg: CT) may be performed on patients, which may increase an amount of radiation to which the patients are exposed.

Various computer vision techniques have been trialed in medical and surgical settings to reconstruct 3D surfaces of target organs including shape-from-shading (SfS), visual simultaneous localization and mapping (SLAM), and Structure from motion (SfM) photogrammetry. SfM is an established computer vision algorithm which enables 3D reconstruction from a collection of two-dimensional (2D) images. SfM is a low-cost, automated technique that uses overlapping images to create 3D models and point clouds of a scene or object by calculating camera positions and scene structure. The benefits of SfM include offline use, ability to process large, nonsequential image stacks, and high resolutions reconstructions. SfM has previously been used to reconstruct various internal organs from endoscopy including the sinus, stomach, and bladder. However, most 3D reconstruction methods including SfM, suffer from scale ambiguity. This limits the potential of SfM for generating laryngotracheal surfaces for quantitative diagnostic purposes, as no information on airway or stenosis diameter can be obtained without ground truth measurements. Further, endoscopic camera parameters that are relied on for SfM vary with each patient exam due to the adjustable focus on camera systems used for clinical bronchoscopy and are not know prior to the clinical exam. An additional problem is that visible anatomical features of laryngotracheal surfaces may be sparse, and therefore not sufficient for SfM algorithms.

### SUMMARY

In one example, the above issues may be addressed via a method, comprising capturing a sequential motion picture of an internal three-dimensional (3D) surface of a patient anatomy using an endoscopic image capturing device; applying a contrast-enhancement algorithm to the captured sequential motion picture to create a contrast-enhanced endoscopic video sequence; reconstructing a 3D surface model of the patient anatomy from the contrast-enhanced endoscopic video sequence using structure from motion (SfM) photogrammetry; scaling the 3D surface model to real-world dimensions of the patient anatomy, to generate a scaled 3D surface model; calculating one or more measurements of the patient anatomy using the scaled 3D surface model; and displaying the one or more measurements on a display device and/or storing the one or more measurements in a memory.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### DESCRIPTION OF DRAWINGS

FIG. 1A illustrates a first block diagram of an example, non-limiting computer environment operable to execute one or more of the various aspects of the present disclosure.

FIG. 1B illustrates a second block diagram of the example, non-limiting computer environment of FIG. 1A.

FIG. 2 illustrates a non-limiting example embodiment of a 3D object reconstruction computing system, according to various aspects of the present disclosure.

FIG. 3 is a schematic diagram illustrating an exemplary general procedure for generating a 3D surface of a larynx and trachea of a patient.

FIG. 4 is a schematic diagram illustrating an exemplary procedure for reconstructing the 3D surface of a larynx and trachea from images acquired via an endoscope, using structure from motion (SfM).

FIG. 5 is a diagram illustrating an exemplary procedure for determining a minimum inscribed sphere diameter of the 3D surface of the larynx and trachea.

FIG. 6 is a flowchart that illustrates a high-level exemplary method for reconstructing the 3D surface of the larynx and trachea, according to various aspects of the present disclosure.

FIG. 7 is a flowchart that illustrates an exemplary method for generating an enhanced contrast sequential motion picture of the larynx and trachea.

FIG. 8 is a flowchart that illustrates an exemplary method for selecting a starting point for performing a reconstruction of the 3D surface of the larynx and trachea.

FIG. 9 is a flowchart that illustrates an exemplary method for scaling a reconstruction of the 3D surface of the larynx and trachea to real world dimensions.

FIG. 10 is an image of the larynx and trachea including vocal cords of the patient.

FIG. 11 is an image of an interior of a laryngoscope inserted into the trachea of the patient.

FIG. 12 is a diagram illustrating a procedure for performing a measurement of an anatomical feature of the larynx and trachea.

### DETAILED DESCRIPTION

Systems and methods are disclosed herein for using structure from motion (SfM) techniques to reconstruct a three-dimensional (3D) surface model of internal surfaces of a patient anatomy, such as a tubular anatomical region of a patient, from clinical endoscopy videos. For the purposes of this disclosure, the systems and methods are described with respect to a laryngotracheal passage of a patient including the larynx and trachea of the patient. However, it should be appreciated that in other embodiments, the disclosed systems and methods may be applied to a different tubular anatomical region without departing from the scope of this disclosure. Results have demonstrated excellent resolution of such reconstructions, when compared to high-resolution CT scans (surface errors <0.300 mm). This technology has immense clinical potential in improving understanding of various diseases, such as, in the case of the laryngotracheal passage, upper airway disease including vocal fold paralysis, laryngeal cancer, subglottic hemangiomas, subglottic stenosis, tracheal stenosis, tracheal sleeves, and tracheomalacia and allows for quantitative analysis of complex laryngotracheal geometries. Presently, quantitative measures of airway caliber and shape, which are critical for diagnostic purposes, are only obtainable via CT and MRI. The disclosed method serves as a cost-effective and radiation-free alternative to advanced imaging through CT or MRI. The SIM reconstructions are obtained from clinical endoscopy which is the gold-standard evaluation method for complex airway disease. The methodology is implementable into clinical workflows.

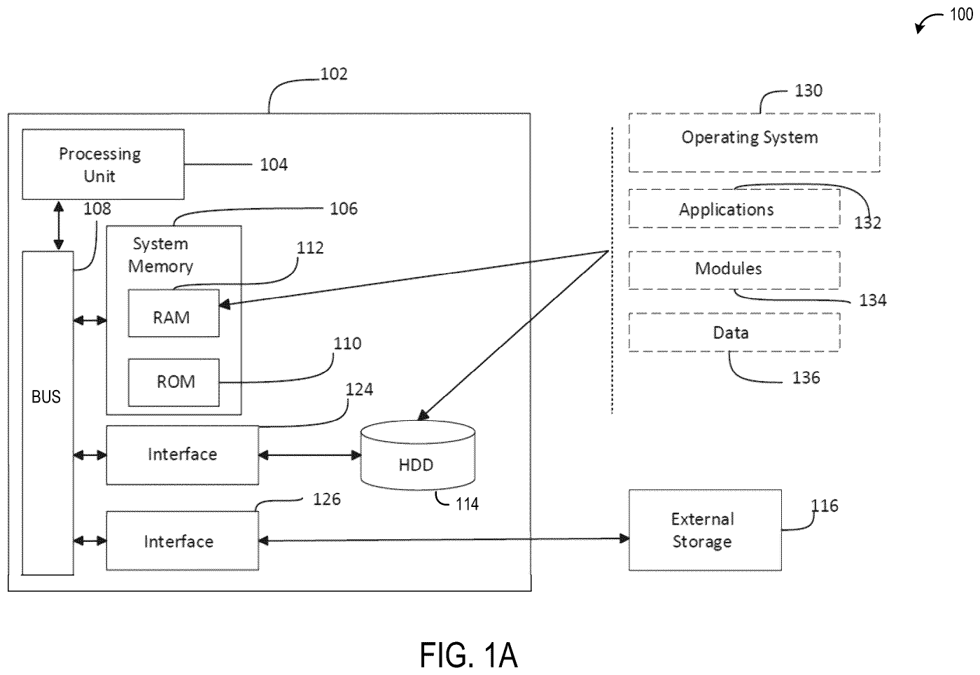

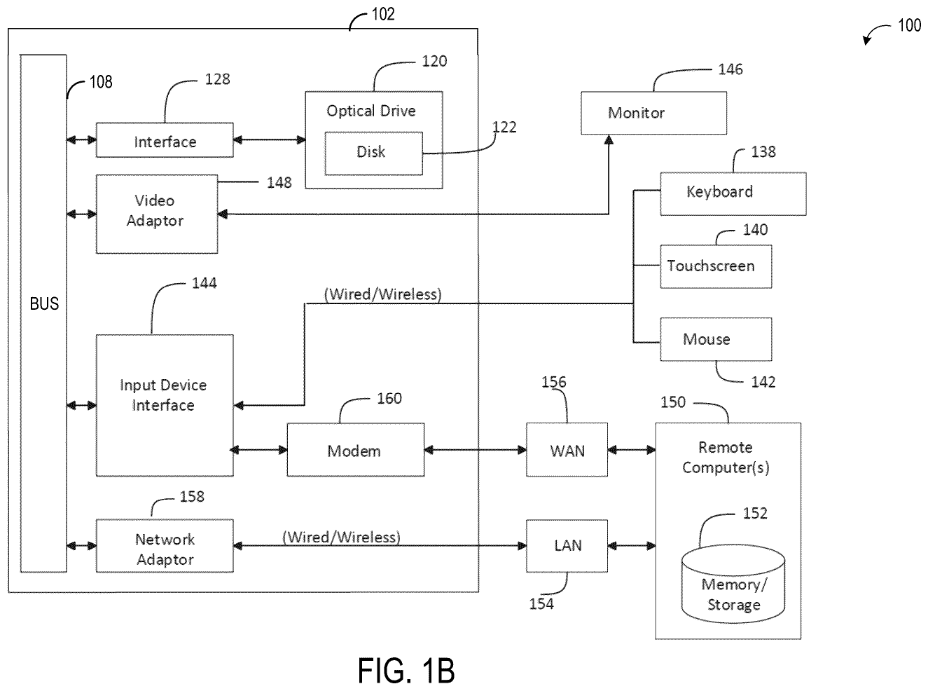

Referring now to the figures, FIGS. 1A and 1B show a block diagram of an example computer architecture 100 that facilitates wireless communications according to one or more embodiments described herein. The computer 100 can provide networking and communication capabilities between a wired or wireless communication network and a server and/or communication device.

To provide additional context for various embodiments described herein, FIGS. 1A and 1B and the following discussion are intended to provide a brief, general description of a suitable computing environment 100 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference to FIGS. 1A and 1B, the example computer architecture 100 includes a computer 102, the computer 102 including a processing unit 104, a system memory 106 and a system bus 108. The system bus 108 couples system components including, but not limited to, the system memory 106 to the processing unit 104. The processing unit 104 can be any of various commercially available processors. Dual microprocessors and other multi-processor architectures can also be employed as the processing unit 104.

The system bus 108 can be any of several types of bus structure that can further interconnect to a memory bus (with or without a memory controller), a peripheral bus, and a local bus using any of a variety of commercially available bus architectures. The system memory 106 includes ROM 110 and RAM 112. A basic input/output system (BIOS) can be stored in a nonvolatile memory such as ROM, erasable programmable read only memory (EPROM), EEPROM, which BIOS contains the basic routines that help to transfer information between elements within the computer 102, such as during startup. The RAM 112 can also include a high-speed RAM such as static RAM for caching data.

The computer 102 further includes an internal hard disk drive (HDD) 114 (e.g., EIDE, SATA), one or more external storage devices 116 (e.g., a magnetic floppy disk drive (FDD), a memory stick or flash drive reader, a memory card reader, etc.) and an optical disk drive 120 (shown in FIG. 1B), which can read or write from disk 122, including but not limited to a CD-ROM disc, a DVD, a BD, etc. While the internal HDD 114 is illustrated as located within the computer 102, the internal HDD 114 can also be configured for external use in a suitable chassis (not shown). Additionally, while not shown in computer architecture 100, a solid state drive (SSD) could be used in addition to, or in place of, an HDD 114. The HDD 114, external storage device(s) 116 and optical disk drive 120 can be connected to the system bus 108 by an HDD interface 124, an external storage interface 126 and an optical drive interface 128 of FIG. 1B, respectively. The interface 124 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 102, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 112, including an operating system 130, one or more application programs 132, other program modules 134 and program data 136. All or portions of the operating system, applications, modules, and/or data can also be cached in the RAM 112. The systems and methods described herein can be implemented utilizing various commercially available operating systems or combinations of operating systems.

Computer 102 can optionally comprise emulation technologies. For example, a hypervisor (not shown) or other intermediary can emulate a hardware environment for operating system 130, and the emulated hardware can optionally be different from the hardware illustrated in FIGS. 1A and 1B. In such an embodiment, operating system 130 can comprise one virtual machine (VM) of multiple VMs hosted at computer 102. Furthermore, operating system 130 can provide runtime environments, such as the Java runtime environment or the .NET framework, for applications 132. Runtime environments are consistent execution environments that allow applications 132 to run on any operating system that includes the runtime environment. Similarly, operating system 130 can support containers, and applications 132 can be in the form of containers, which are lightweight, standalone, executable packages of software that include, e.g., code, runtime, system tools, system libraries and settings for an application. Further, computer 102 can be enable with a security module, such as a trusted processing module (TPM). For instance with a TPM, boot components hash next in time boot components, and wait for a match of results to secured values, before loading a next boot component. This process can take place at any layer in the code execution stack of computer 102, e.g., applied at the application execution level or at the operating system (OS) kernel level, thereby enabling security at any level of code execution.

A user can enter commands and information into the computer 102 through one or more wired/wireless input devices depicted in FIG. 1B, e.g., a keyboard 138, a touch screen 140, and a pointing device, such as a mouse 142. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 104 through an input device interface 144 that can be coupled to the system bus 108, but can be connected by other interfaces, such as a parallel port, an IEEE 1394 serial port, a game port, a USB port, an IR interface, a BLUETOOTH® interface, etc.

A monitor 146 or other type of display device can be also connected to the system bus 108 via an interface, such as a video adapter 148. In addition to the monitor 146, a computer typically includes other peripheral output devices (not shown), such as speakers, printers, etc.

The computer 102 can operate in a networked environment using logical connections via wired and/or wireless communications to one or more remote computers, such as a remote computer(s) 150. The remote computer(s) 150 can be a workstation, a server computer, a router, a personal computer, portable computer, microprocessor-based entertainment appliance, a peer device or other common network node, and typically includes many or all of the elements described relative to the computer 102, although, for purposes of brevity, only a memory/storage device 152 is illustrated. The logical connections depicted include wired/wireless connectivity to a local area network (LAN) 154 and/or larger networks, e.g., a wide area network (WAN) 156. Such LAN and WAN networking environments are commonplace in offices and companies, and facilitate enterprise-wide computer networks, such as intranets, all of which can connect to a global communications network, e.g., the Internet.

When used in a LAN networking environment, the computer 102 can be connected to the local network 154 through a wired and/or wireless communication network interface or adapter 158. The adapter 158 can facilitate wired or wireless communication to the LAN 154, which can also include a wireless access point (AP) disposed thereon for communicating with the adapter 158 in a wireless mode.

When used in a WAN networking environment, the computer 102 can include a modem 160 or can be connected to a communications server on the WAN 156 via other means for establishing communications over the WAN 156, such as by way of the Internet. The modem 160, which can be internal or external and a wired or wireless device, can be connected to the system bus 108 via the input device interface 144. In a networked environment, program modules depicted relative to the computer 102 or portions thereof, can be stored in the remote memory/storage device 152. It will be appreciated that the network connections shown are example and other means of establishing a communications link between the computers can be used.

When used in either a LAN or WAN networking environment, the computer 102 can access cloud storage systems or other network-based storage systems in addition to, or in place of, external storage devices 116 as described above. Generally, a connection between the computer 102 and a cloud storage system can be established over a LAN 154 or WAN 156 e.g., by the adapter 158 or modem 160, respectively. Upon connecting the computer 102 to an associated cloud storage system, the external storage interface 126 can, with the aid of the adapter 158 and/or modem 160, manage storage provided by the cloud storage system as it would other types of external storage. For instance, the external storage interface 126 can be configured to provide access to cloud storage sources as if those sources were physically connected to the computer 102.

The computer 102 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the disclosed subject matter has been described in connection with various embodiments and corresponding Figures, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors can exploit nano-scale architectures such as, but not limited to, molecular and quantum-dot based transistors, switches and gates, in order to optimize space usage or enhance performance of user equipment. A processor may also be implemented as a combination of computing processing units.

In the subject specification, terms such as “store,” “storage,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can include both volatile and nonvolatile memory.

As used in this application, the terms “component,” “system,” “platform,” “layer,” “selector,” “interface,” and the like are intended to refer to a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media, device readable storage devices, or machine-readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor, wherein the processor can be internal or external to the apparatus and executes at least a part of the software or firmware application. As yet another example, a component can be an apparatus that provides specific functionality through electronic components without mechanical parts, the electronic components can include a processor therein to execute software or firmware that confers at least in part the functionality of the electronic components.

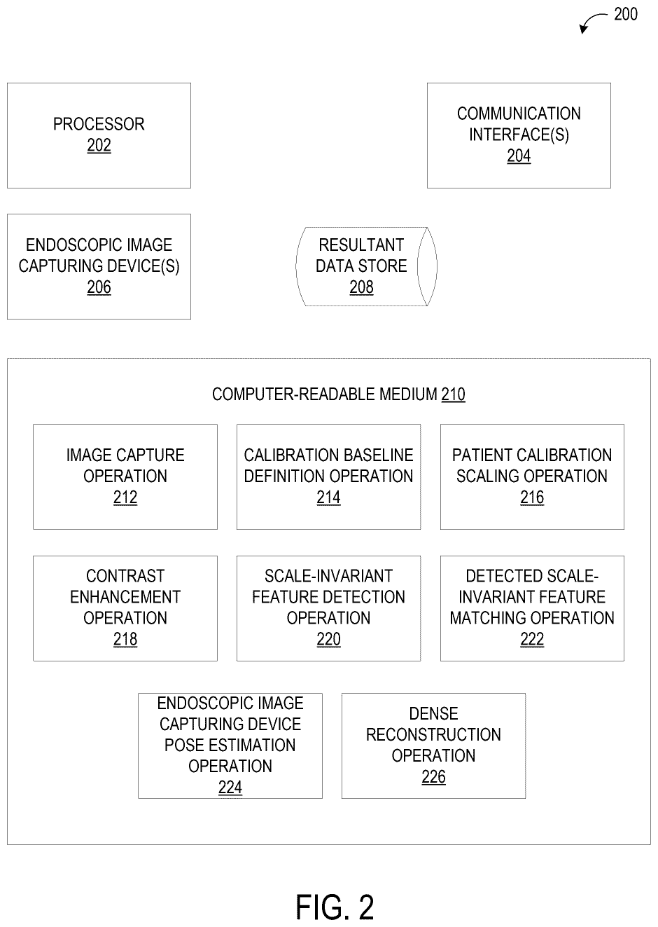

Referring now to FIG. 2, herein described is a block diagram illustrating a non-limiting example embodiment of a 3D object-from-motion reconstruction computing system 200, according to various aspects of the present disclosure. The illustrated 3D object-from-motion reconstruction computing system 200 may be implemented by any computing device or collection of computing devices, including but not limited to a desktop computing device, a laptop computing device, a mobile computing device, a server computing device, a computing device of a cloud computing system, and/or combinations thereof. In some embodiments, at least a portion of the 3D object-from-motion reconstruction computing system 200 may be implemented using a smartphone.

As shown, the 3D object-from-motion reconstruction computing system 200 includes one or more processors 202, one or more communication interfaces 204, one or more endoscopic image capturing devices 206, a resultant data store 208, and a computer-readable medium 210.

In some embodiments, the one or more processors 202 may include any suitable type of general-purpose computer processor. In some embodiments, the one or more processors 202 may include one or more special-purpose computer processors or AI accelerators optimized for specific computing tasks, including but not limited to graphical processing units (GPUs), vision processing units (VPTs), and tensor processing units (TPUs).

In some embodiments, the one or more communication interfaces 204 include one or more hardware and or software interfaces suitable for providing communication links between components. The one or more communication interfaces 204 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

In some embodiments, the computer-readable medium 210 has stored thereon logic that, in response to execution by the one or more processors 202, cause the 3D object reconstruction computing system 200 to provide an image capture operation 212, a calibration baseline definition operation 214, a patient calibration scaling operation 216, a contrast-enhancement operation 218, a scale-invariant feature detection operation 220, a detected scale-invariant feature matching operation 222, an endoscopic image capturing device pose estimation operation 224, and a dense reconstruction operation 226.

As used herein, “operation” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, MATLAB™, and Python. An operation may be compiled into executable programs or written in interpreted programming languages. Software operations may be callable from other operations or from themselves. Generally, the operations described herein refer to logical modules that can be merged with other operations, or can be divided into sub-operations. The operations can be implemented by logic stored in any type of computer-readable medium or computer storage device and be stored on and executed by one or more general purpose computers, thus creating a special-purpose computer configured to provide the engine or the functionality thereof. The operations can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

In some embodiments, the image capture operation 212 is configured to receive one or more images captured by the one or more endoscopic image capturing devices 206. In some embodiments, the image capture operation 212 receives one or more images of a calibration object. In one embodiment, the calibration object is a checkerboard. In one embodiment, the calibration object is a 3D or 2D object with a pattern of known physical spacing used for calculation of optical parameters. In one embodiment, the one or more endoscopic image capturing devices 206 comprises an endoscope. In one embodiment, the endoscope comprises a lens, a light source, and an image sensor, such as a camera. In one embodiment, the one or more endoscopic image capturing devices 206 comprises an endoscope and image capture system used for clinical settings, such as endoscopy. In one embodiment, the endoscope and image capture system is rigid. In one embodiment, the endoscope and image capture system is flexible.

In some embodiments, a calibration baseline definition operation 214 receives the captured one or more images of the calibration object from the image capture operation 212. In some embodiments, the calibration baseline definition operation 214 generates a calibration baseline matrix (including but not limited to focal length, image center, and distortion coefficients) that is then stored in the resultant data store 208. The calibration baseline matrix removes optical distortion generated by the image capturing devices 206.

In some embodiments, the image capture operation 212 is configured to receive a sequential motion picture from the one or more endoscopic image capturing devices 206. In one embodiment, the sequential motion picture of the 3D object to be reconstructed is captured. In one embodiment, the sequential motion picture is a continuous video captured with a single pass (motion) of the one or more endoscopic image capturing device 206. In one embodiment, the 3D object to be reconstructed is a patient's airway. In one embodiment, the sequential motion of the 3D object to be reconstructed also includes (captures) the patient calibration scaling device.

In some embodiments, a patient calibration scaling operation 216 receives the captured sequential motion picture from the image capture operation 212. In some embodiments, the patient calibration scaling operation 216 generates or defines a patient calibration scaling for the captured sequential motion picture. The patient calibration scaling defines the scaling from image space to physical/world space (pixels to meters). In one embodiment, the patient calibration scaling device is a physical object of known physical dimensions placed near the 3D object to be reconstructed. In one embodiment, the patient calibration scaling device is a laryngoscope.

In some embodiments, a contrast-enhancement operation 218 receives the captured sequential motion picture from the patient calibration scaling operation 216. In some embodiments, a contrast-enhancement algorithm is applied to the captured sequential motion picture to create a contrast-enhanced endoscopic video sequence.

In some embodiments, a scale-invariant feature detection operation 220 receives the contrast-enhanced endoscopic video sequence from the contrast-enhancement operation 218. In some embodiments, one or more scale-invariant features are detected from one or more images of the contrast-enhanced endoscopic video sequence. In one embodiment, each image is acquired from a different time-point. In one embodiment, a first image is at a first time point, and a second image is at a second time point. In the one embodiment, the first time point and the second time point are different. In one embodiment, the one or more images comprise an additional third image with a third time point. In one embodiment, the third time point is different from the first time point and the second time point. In one embodiment, one-thousand or more images can be obtained from the contrast-enhanced endoscopic video sequence and have features detected in them.

In some embodiments, a detected scale-invariant feature matching operation 222 receives the detected one or more scale-invariant features of each image from the scale-invariant feature detection operation 220 to create matched one or more scale-invariant features. In some embodiments, the detected one or more scale-invariant features of each image are matched to the detected one or more scale-invariant features of the sequentially neighboring images (e.g., the next 20 images). In one embodiment, the detected one or more scale-invariant features of each image are matched to the detected one or more scale-invariant features of every other image in the contrast enhanced, sequentially captured images. In one embodiment, the detected one or more scale-invariant features of each image are matched to the detected one or more scale-invariant features of a manually defined list of images.

In some embodiments, an endoscopic image capturing device pose estimation operation 224 receives the one or more of the following: the one or more images of the sequential motion picture, the matched one or more scale-invariant features from the detected scale-invariant feature matching operation 222, and the optical parameters of the calibration baseline definition operation 214. In some embodiments, the endoscopic image capturing device pose estimation operation 224 estimates one or more poses of the one or more endoscopic image capturing devices 206.

In some embodiments, a dense reconstruction operation 226 receives the matched one or more scale-invariant features from the detected scale-invariant feature matching operation 222 scale-invariant, and the one or more poses of the one or more endoscopic image capturing devices 206 from the endoscopic image capturing device pose estimation operation 224. In some embodiments, the dense reconstruction operation 226 combines the mapped one or more scale-invariant features with the one or more poses of the one or more endoscopic image capturing devices 206. In one embodiment, the 3D object is reconstructed from the combining of the mapped one or more scale-invariant features with the one or more poses of the one or more endoscopic image capturing devices 206.

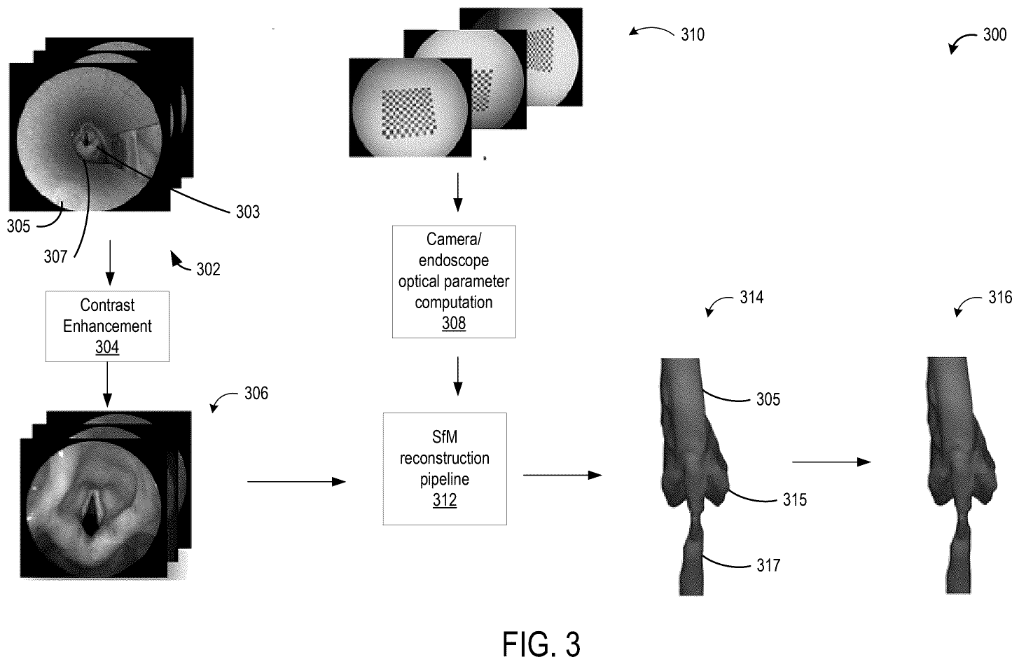

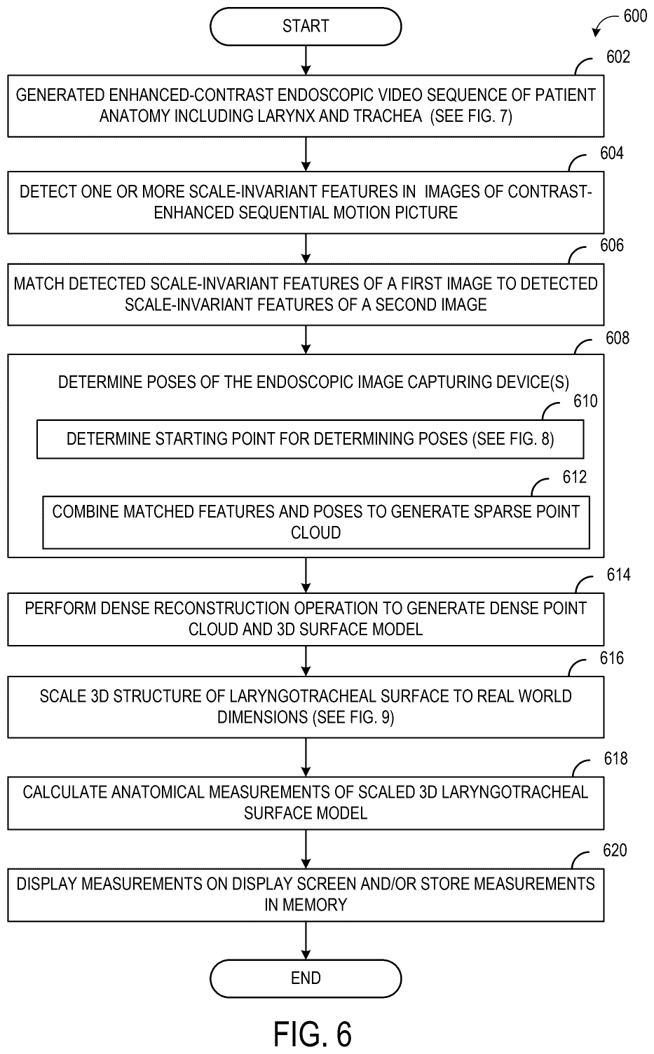

One or more of the operations described above in reference to FIG. 2 may be performed in accordance with methods described herein in reference to FIGS. 6-9, to generate a 3D surface model of a larynx and trachea of a patient from clinical endoscopy videos, using SfM techniques and without relying on imaging modalities such as CT, MRI, etc. A high-level overview of the general procedure is first provided in reference to FIGS. 3-5.

Turning now to FIG. 3, a first schematic diagram 300 depicts various stages of a general procedure for generating a 3D surface model of a larynx and trachea of a patient. At a first stage of the general procedure, an endoscopic video sequence 302 of an anatomy 303 of the patient including the laryngoscope, larynx, and trachea is generated using an endoscope inserted into a laryngoscope 305 positioned in a throat of the patient. Initial frames of the endoscopic video sequence 302 include views of the laryngoscope 305; frames acquired after the endoscope passes a bottom edge 307 of the laryngoscope 305 do not include views of the laryngoscope 305. Frames including views of the laryngoscope 305 may be used to scale the 3D surface model, as described in greater detail below.

In a second stage of the general procedure, a contrast enhancement algorithm 304 is applied to each frame of the endoscopic video sequence 302 to increase a contrast of the anatomy 303 including the larynx and the trachea. A resulting contrast-enhanced endoscopic video sequence 306 is then input into an SfM reconstruction pipeline 312, which reconstructs a 3D surface model 314 of the anatomy 303. The 3D surface model may comprise a larynx portion 315 and a trachea portion 317. An additional input into the SfM reconstruction pipeline 312 may include a set of optical parameters of the endoscope and one or more cameras positioned thereupon determined at a camera/endoscope optical parameter computation stage 308. The set of optical parameters may be calculated using a plurality a calibration images (e.g., a video sequence) 310 of a calibration target at different angles. In various examples, the calibration target is a high-resolution ceramic checkerboard. In other examples, a different calibration target may be used.

The 3D surface model 314 may not be generated at a scale identical to the actual larynx and trachea of the patient. Therefore, after the 3D surface model 314 is generated via the SfM reconstruction pipeline 312, the 3D surface model 314 may be scaled to real-world dimensions of the patient via a scaling algorithm, to generate a scaled 3D surface model 316. The scaled 3D surface model 316 may then be used to perform quantitative measurements of the anatomy 303, including dimensions and distances between features of the larynx portion 315 and the trachea portion 317.

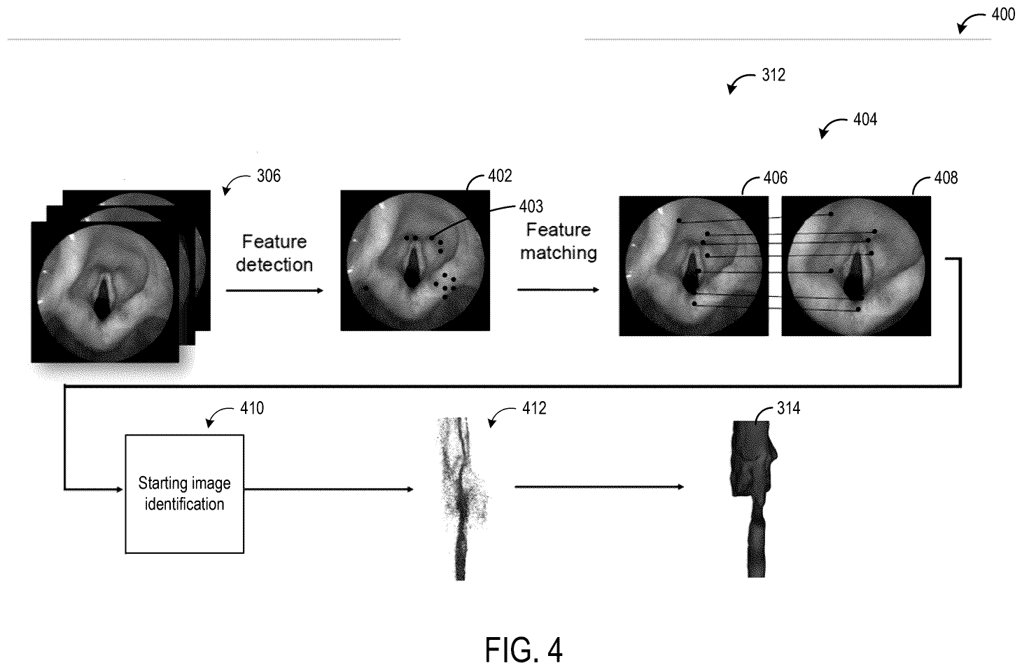

FIG. 4 shows a second schematic diagram 400 depicting stages of the SfM reconstruction pipeline 312 of FIG. 3. During a first feature detection stage 402 of the SfM reconstruction pipeline 312, a feature detection algorithm is applied to each image the contrast-enhanced endoscopic video sequence 306 generated by the contrast enhancement algorithm, to identify and mark a plurality of anatomical features 403 of the anatomy 303. After the anatomical features 403 have been identified and marked in the images of the contrast-enhanced endoscopic video sequence 306, a second, feature matching stage of the SfM reconstruction pipeline 312 is performed. During the second, feature matching stage 404, the anatomical features 403 of each image 406 of the feature-marked contrast-enhanced endoscopic video sequence 306 are matched to corresponding anatomical features 403 of one or more subsequent images 408 of the feature-marked contrast-enhanced endoscopic video sequence 306.

At a third stage 410, a starting image of the feature-matched contrast-enhanced endoscopic video sequence 306 is identified. In various examples, the starting image may be an image in which a subglottis of the patient is detected, as described in greater detail below. After the starting image is identified, an SfM reconstruction 412 is performed on the feature-matched contrast-enhanced endoscopic video sequence 306 starting at the starting image. The SfM reconstruction 412 generates the 3D surface model 314.

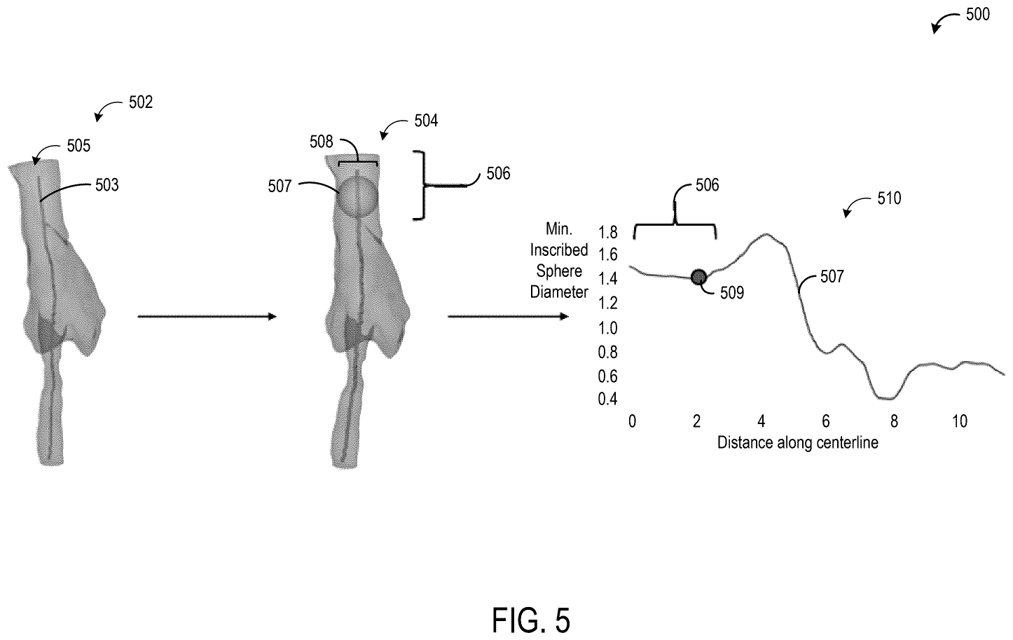

FIG. 5 shows a third schematic diagram 500 illustrating a scaling procedure for scaling the 3D surface model 314 to generate the scaled 3D surface model 316 of FIG. 3. At a first stage 502 of the scaling procedure, a centerline 503 of an airway 505 within an upper portion 506 of the 3D surface model 314 is computed, using techniques known in the art. The upper portion 506 may correspond to a length of the laryngoscope 305 (e.g., a portion of the 3D surface model 314 occupied by the laryngoscope 305). At a second stage 504, an internal diameter of the laryngoscope 305 is calculated, which may be equivalent to a diameter 508 of a smallest inscribed sphere 507 in the airway 505 along the centerline 503. A graph 510 shows a plot 507 of inscribed sphere diameters (on the y axis) as a function of distance along the centerline 503 (on the x axis). A local minimum 509 of plot 507 within the upper portion 506 indicates the diameter 508 of the smallest inscribed sphere 507. A scaling factor may then be retrieved from a lookup table of minimum internal diameters of physical laryngoscopes, based on the internal diameter of the laryngoscope 305, and the scaling factor may be used to scale the 3D surface model 314 to generate the scaled 3D surface model 316.

Referring now to FIG. 6, an exemplary high-level method 600 is shown for reconstructing a 3D surface model of a larynx and trachea of a patient from clinical endoscopy videos, using SfM techniques, as summarized above in reference to FIGS. 3-5. Method 600 and the other methods described herein may be performed by a processor of a 3D object-from-motion reconstruction computing system, such as the processor 202 of the 3D object-from-motion reconstruction computing system 200 of FIG. 2, based on instructions stored in a memory of the 3D object-from-motion reconstruction computing system 200 (e.g., system memory 106 of FIGS. 1A and 1B).

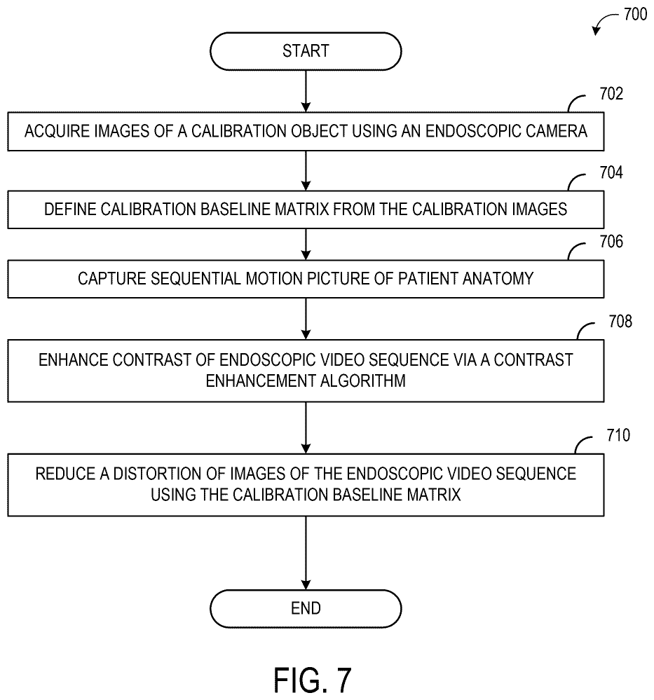

At 602, method 600 begins with generating an enhanced-contrast endoscopic video sequence of an anatomical portion of a patient including a larynx and a trachea of the patient (e.g., contrast-enhanced endoscopic video sequence 306 of FIG. 3). A contrast enhancement operation (e.g., contrast enhancement operation 218 of FIG. 2) may be applied to a sequential motion picture acquired by an endoscope inserted into a laryngoscope positioned in a patient's airway to create the contrast-enhanced endoscopic video sequence. The contrast-enhanced endoscopic video sequence may be captured using one or more endoscopic image capturing devices, such as a camera. The contrast enhancement operation may increase a visibility of features within the captured images, which may facilitate a more accurate detection of scale-invariant features in subsequent steps. For example, when capturing endoscopic video of a patient's airway, the contrast enhancement algorithm may increase a visual distinction between different tissue structures, making anatomical features more readily identifiable. The generation of the contrast-enhanced endoscopic video sequence is described in greater detail below in reference to FIG. 7.

At 604, method 600 includes detecting one or more scale-invariant features in images of the contrast-enhanced endoscopic video sequence (e.g., scale-invariant feature detection operation 220 of FIG. 2). The scale-invariant features are distinctive features that remain recognizable despite changes in scale, rotation, or perspective. The scale-invariant features may serve as reference points that can be tracked across a plurality of images acquired from different time points in the endoscopic video sequence. In some implementations, the system may analyze hundreds or thousands of images from the contrast-enhanced endoscopic video sequence, identifying scale-invariant features in each image that can be matched across the sequence.

At 606, method 600 includes matching the detected scale-invariant features of each image of the contrast-enhanced endoscopic video sequence with subsequent images of the contrast-enhanced endoscopic video sequence (e.g., scale-invariant feature matching operation 222 of FIG. 2). This matching process creates pairs of corresponding features across image sequences of the video sequence that represent the same physical point viewed from different perspectives or at different times. The system may match the detected scale-invariant features of each image to the detected scale-invariant features of a plurality of sequentially neighboring images. For example, the system may match scale-invariant features of a first image with 35 subsequent, consecutive images in the sequence, or a different number. Alternatively, the system may match features between every image in the sequence or between images specified in a manually defined list. For example, the system may track specific tissue landmarks across a plurality of frames as the endoscope moves through patient anatomy (e.g., anatomy 303). The matching images may be stored in a database, such as resultant data store 208 of FIG. 2.

At 608, method 600 includes determining a pose of the endoscopic image capturing device for each image in the endoscopic video sequence (e.g., pose estimation operation 224 of FIG. 2), where the pose includes a position and an orientation of the endoscopic image capturing device, and generating a sparse point cloud representation of a laryngotracheal surface of the patient (e.g., sparse reconstruction). Determining the poses of the endoscopic image capturing device may rely on the matched scale-invariant features along with optical parameters obtained during the calibration process described in reference to FIG. 7 (e.g., the calibration baseline matrix). For example, the optical parameters may include a focal length of an endoscopic camera, a center of an image, and/or distortion coefficients determined using calibration images of a calibration object such as a checkerboard. By calculating how the matched features change position between images in accordance with the camera poses, the system can infer the movement of the camera between those frames, effectively reconstructing a path of the endoscopic camera(s) through the anatomy.

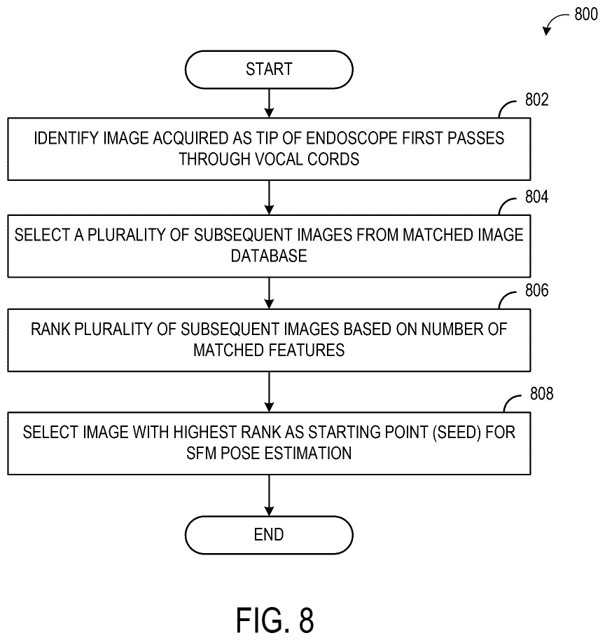

At 610, determining the pose of the endoscopic image capturing device for each image in the endoscopic video sequence and defining a sparse point cloud further comprises determining a starting point for the SfM algorithm. In other words, a starting image may be selected to serve as the starting point or seed for the pose estimation. In one example, the starting image is an image acquired as the tip of the endoscope first passes through specific anatomical landmarks, such as the vocal cords. The selection of the starting image is described below in reference to FIG. 8.

At 612, method 600 includes combining the matched scale-invariant features with the corresponding poses of the endoscopic image capturing device to generate a sparse point cloud representation of a 3D structure of a laryngotracheal surface of the patient (e.g., the sparse reconstruction). In particular, the calibration baseline matrix generated as described in reference to FIG. 7 is used to enable pose estimation and to triangulate matched features into 3D, meaning, the matrix may define how points map from 2D image space to 3D world space (e.g., how matched 2D features from step 606 are projected back into 3D, along with the pose locations). The camera poses and the 3D feature locations may be solved in an iterative fashion. For example, the algorithm may start with two images, determine the pose between them, project matched points into 3D, and then solve for poses of new images and project those points into 3D, and so on, until all the images are processed. In this way, the model is grown from the starting seed image until all images in the stack are processed (pose calculated and features projected to 3D).

Proper camera calibration is crucial to obtaining an anatomically accurate 3D representation or model. The calibration matrix determines how features (detected in 2D) are projected in 3D. As a result, errors in the matrix may lead to distortion of the 3D model where the relationship between width and depth of 3D objects or features will not be correct. For a tool with potential usage in clinical diagnostic (eg: airway and stenosis sizing), obtaining the correct 3D morphology and shape is crucial. In other words, the sparse reconstruction process leverages the known camera positions and orientations to triangulate the 3D positions of points visible in multiple images, creating a detailed representation of the surface geometry of the anatomy. This combination process may involve sophisticated algorithms that optimize the 3D positions of features while minimizing reprojection errors.

At 614, method 600 includes performing a dense reconstruction operation on the endoscopic video sequence (e.g., dense reconstruction operation 226 of FIG. 2) to generate a dense point cloud, from which a 3D laryngotracheal surface model (e.g., 3D surface model 314 of FIG. 3) of the larynx and trachea of the patient may be created. During this operation, a sparse set of matched features may be transformed into a comprehensive 3D model by computing the spatial coordinates of many additional points in the scene. Starting with the known camera poses and sparse point cloud, a depth map is computed for each image, which defines the depth of every pixel in the image. The depth maps (for each image) and the known poses are then fused together to create the dense 3D point cloud. Various algorithms may be used for computing image depth maps and dense reconstruction, including trained neural networks. In one example, the MutliView Stereo (MVS) algorithm is used. Once the dense point cloud is created, a surface mesh is generated from the dense point cloud, using one of various 3D-point-to-surface-meshing algorithms (e.g., Poisson, Deluanay, etc.). The resulting 3D laryngotracheal surface model represents the geometry of the captured anatomy, such as the larynx and trachea, in arbitrary measurement units.

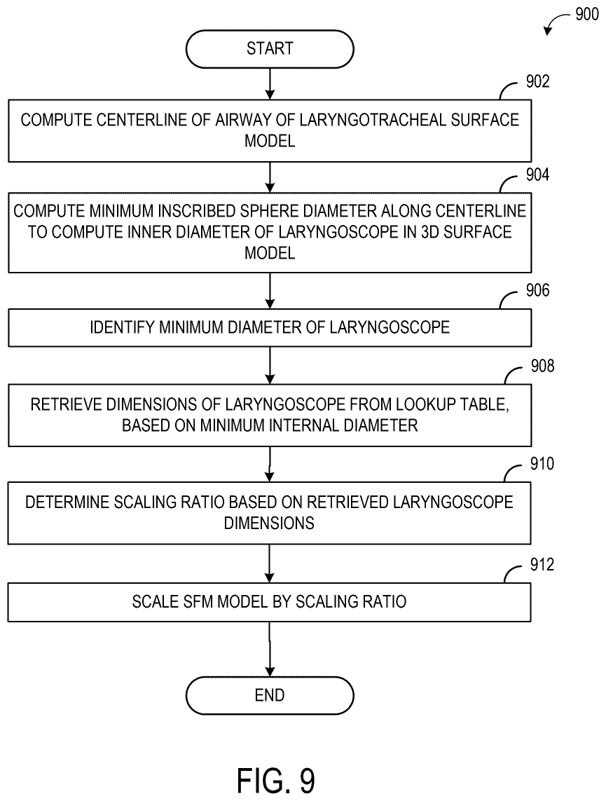

At 616, method 600 includes scaling the 3D laryngotracheal surface model to real-world dimensions. This scaling process may rely on a patient calibration scaling device of known physical dimensions that was captured in the endoscopic video sequence, such as the laryngoscope. The scaling of the 3D laryngotracheal surface model is described in greater detail below in reference to FIG. 9.

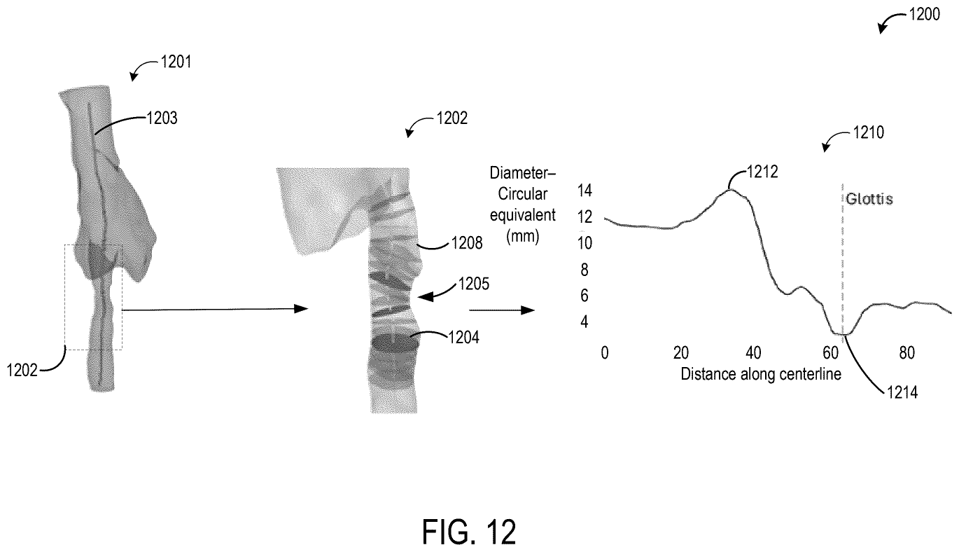

At 618, method 600 includes calculating anatomical measurements of the scaled 3D laryngotracheal surface model. A quantitative analysis may be performed on the scaled 3D laryngotracheal surface model to extract clinically relevant measurements, such as a caliber of an airway of the laryngotracheal surface (e.g., airway 505 of FIG. 5), shape parameters, distances between anatomical landmarks, cross-sectional areas at various points along the airway, and the like. The quantitative data may provide diagnostic information for conditions such as vocal fold paralysis, laryngeal cancer, subglottic hemangiomas, subglottic stenosis, tracheal stenosis, tracheal cartilaginous sleeves, complete tracheal rings, and tracheomalacia. For example, the system may calculate a minimum cross-sectional area in a patient with suspected airway stenosis, providing physicians with objective measurements for which a CT or MRI scan may be relied on. By generating the objective measurements via the 3D object-from-motion reconstruction computing system rather than from a CT or MRI image, the patient may avoid exposure to radiation, and use of imaging resources of a hospital may be reduced and managed more efficiently. An example of using the scaled 3D laryngotracheal surface model for taking measurements is described below in reference to FIG. 12.

At 620, method 600 includes displaying the measurements on a display device (e.g., monitor 146 of computer architecture 100 of FIGS. 1A and 1B) and/or storing the measurements in the memory for future reference, comparison, and/or analysis. This storage ensures that the quantitative data derived from the 3D reconstruction remains accessible for clinical decision-making, research purposes, or longitudinal patient monitoring. The stored measurements may provide a permanent record that can be integrated into the patient's medical history and compared with future assessments to track disease progression or treatment response.

In this way, method 600 provides a comprehensive framework for generating accurate 3D reconstructions of anatomical structures from endoscopic video sequences. By leveraging SfM techniques with specialized enhancements for medical applications, the method enables quantitative analysis of complex laryngotracheal geometries without requiring radiation exposure from imaging modalities. The resulting 3D models and measurements offer clinicians valuable diagnostic information that was previously difficult or impossible to obtain from standard endoscopic examinations alone.

Referring now to FIG. 7, an exemplary method 700 is shown for generating an enhanced-contrast endoscopic video sequence of an anatomical portion of a patient including a larynx and a trachea of the patient. In various examples, method 700 is performed as a part of method 600 of FIG. 6 described above.

At 702, method 700 includes acquiring a plurality of images of a calibration object, using an endoscopic camera. The calibration object may be a 3D anatomic object with known dimensions, for establishing baseline optical parameters that will be used in the reconstruction process described above in reference to FIG. 6. For example, the endoscope with camera head may be calibrated using a 15×15 mm checkerboard. In one example, with the calibration target remaining fixed in place, a video is acquired of the calibration target from multiple angles (e.g., top, bottom, right, and left). The tip of the endoscope may be maintained relatively close to the target, while the camera end is moved like a reverse pendulum or joystick. The parameters obtained in this manner may be used for mapping 2D images captured by the endoscopic camera to corresponding 3D positions in space.

At 704, method 700 includes defining a calibration baseline matrix based on the acquired images of the calibration object. The calibration baseline matrix establishes a set of optical parameters of the endoscopic camera that will be used throughout the reconstruction process. The optical parameters included in the calibration baseline matrix may include, as a non-limiting list, a focal length of the endoscopic camera, a center of an image, and/or distortion coefficients determined using calibration images of the calibration object. In other words, a mathematical model of how the endoscopic camera captures and projects 3D scenes onto 2D images is created, accounting for lens distortion and other optical effects specific to the endoscopic equipment being used. The calibration baseline matrix serves as a reference point for all subsequent image processing and 3D reconstruction operations, ensuring that measurements and spatial relationships derived from the endoscopic images are accurate and consistent.

At 706, method 700 includes capturing a sequential motion picture of a patient anatomy (e.g., anatomy 303) including a laryngotracheal surface using the endoscopic camera. The sequential motion picture may include an object of a known diameter, such as a laryngoscope positioned in a throat of the patient. For example, the endoscopy may be performed by passing through a suspended Parsons 3 laryngoscope, through a stenosis, to a carina of the patient. The sequential motion picture may provide raw visual data from which the 3D reconstruction will be derived, capturing the internal anatomy of the patient's airway from multiple viewpoints as the endoscope moves through the laryngotracheal passage.

At 708, method 700 includes enhancing a contrast of the sequential motion picture of the laryngotracheal surface. A contrast enhancement operation (e.g., contrast enhancement operation 218) may be applied to the sequential motion picture to create a contrast-enhanced endoscopic video sequence. The contrast enhancement may increase a visibility of features within the captured images, which may facilitate a more accurate detection of scale-invariant features in subsequent steps. For example, when capturing endoscopic video of a patient's airway, the contrast enhancement algorithm may increase visual distinction between different tissue structures, making anatomical features more readily identifiable. This enhanced contrast may increase an accuracy of the feature detection and matching processes that form the foundation of the SfM reconstruction technique.

At 710, method 700 includes reducing a distortion of images of the contrast-enhanced endoscopic video sequence using the calibration baseline matrix. That is, the calibration baseline matrix may be used to compute a new image with radial distortion removed. Removing the distortion from each image may lead to more stable SfM reconstructions. Additionally, after the images are undistorted, if a new baseline calibration matrix is generated and undistorted images are reconstructed using SfM, then the new undistorted calibration matrix may be used during reconstruction. In other words, the camera projection function that defines the raw distorted images have radial distortion values, in addition to focal length and camera center values. Once an image has been undistorted, it no longer is defined with radial distortion, so the camera projection function becomes much simpler, with only focal length and camera center coordinates. The simpler camera model is known as a pinhole camera model. After the images are undistorted, the projection of 2D points to 3D is defined with the pinhole camera model. The simpler camera model is determined from the undistortion process, and doesn't require any new data.

Referring now to FIG. 8, an exemplary method 800 is shown for selecting a starting image for performing the pose estimation described in method 600. The method starts at 802, where method 800 includes identifying a first image acquired as a tip of the endoscope first passes through an anatomical region of interest that coincides with roughly a middle of the contrast-enhanced endoscopic video sequence. For example, for an endoscopic video sequence of the laryngotracheal passage as described herein, vocal cords of the patient may be used, as the subglottis is a highly important anatomical region of the laryngotracheal passage that is located at the middle of the contrast-enhanced endoscopic video sequence. For other endoscopic video sequences of other tubular anatomical structures, a different anatomical region of interest may be used.

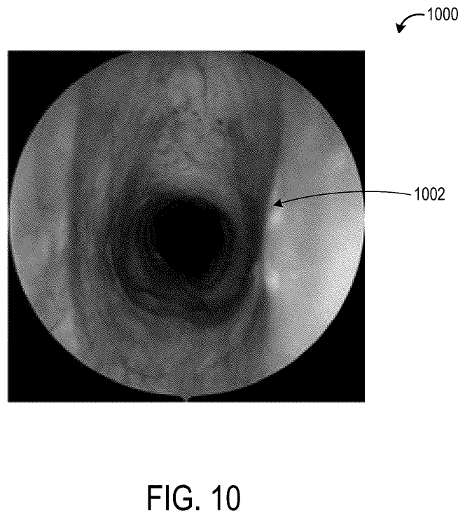

The (contrast enhanced) endoscopic video sequence may be analyzed to locate the specific frame where the endoscope tip initially traverses the vocal cords. For example, when examining a pediatric airway, the first image may include a characteristic appearance of vocal folds at the periphery of the endoscopic view, indicating the transition from supraglottic to subglottic regions. An exemplary first image 1000 is shown in FIG. 10, where vocal cords 1002 are identifiable just at an edge of first image 1000.

At 804, method 800 includes selecting a plurality of subsequent images to the first image from a matched image database such as resultant data store 208, or a different database. The matched image database may be populated with images generated from the feature matching procedure described above in reference to FIG. 6. The selected subsequent images may have strong feature correspondence, which may increase a stability and accuracy of a subsequent reconstruction. The number of subsequent images selected may be predetermined based on empirical testing, or may be dynamically adjusted based on the quality and characteristics of the specific endoscopic video sequence. For instance, the system might select 20-30 sequential frames that show the progression of the endoscope through the subglottic region.

At 806, method 800 includes ranking the plurality of subsequent images based on a number of matched features in each image of the plurality of subsequent images. In other words, each selected image may be assigned a ranking based on the quantity of scale-invariant features of the selected image that have been mapped to the same scale-invariant features appearing in all of the other images of the plurality of subsequent images. Images with more matched features may provide more reliable reference points for the SfM reconstruction algorithm. This ranking process helps identify frames that contain rich visual information about the airway anatomy, such as distinctive tissue textures, anatomical landmarks, or surface variations that can be tracked across multiple viewpoints.

At 808, method 800 includes selecting an image with a highest rank from a predetermined region of interest as a starting point (seed) for the SfMpose estimation described above in reference to FIG. 6. This strategic selection optimizes the reconstruction by beginning with the image of the plurality of subsequent images that includes the most robust set of matched features, thereby establishing a strong foundation for the progressive building of the 3D surface model. Starting with this optimal seed image increases the likelihood of successful feature tracking throughout the sequence and reduces the potential for reconstruction errors or gaps in the resulting 3D surface. Additionally, selecting an image from the middle of the image stack may decreases an error or drift in the reconstruction. Spatial reconstruction error accumulates the further the reconstruction grows from the starting image, so an overall error in the end 3D surface is reduced by selecting an image roughly equidistant from the start and end of the image sequence. For example, in a pediatric subglottic stenosis case, the seed image might be one that clearly shows the narrowest point of the stenosis with multiple identifiable tissue features that can be tracked as the endoscope moves through the airway. The image with the greatest number of corresponding matched features from the overall stack may occur near the beginning when the laryngoscope is in view, but by starting with an image that is acquired within the sub-glottis, the total error may be reduced.

To further clarify, it should be appreciated that the image with greatest number of matches from the entire image stack may not be the most ideal starting image. For example, the image with greatest number of matches from the plurality of subsequent images may be an image from inside the laryngoscope. Rather, a subset of images from the plurality of subsequent images is selected that both includes the subglottis and is near a center of the video sequence. From this subset, the image with the greatest number of matches may be selected.

Referring now to FIG. 9, an exemplary method 900 is shown for scaling a 3D laryngotracheal surface model of a patient, such as 3D surface model 314 of FIG. 3, that is generated by the 3D object-from-motion reconstruction computing system 200 as described herein. In various examples, method 900 may be performed as part of method 600 described above in reference to FIG. 6.

At 902, method 900 includes computing a centerline of an airway of the 3D laryngotracheal surface model. The centerline provides a reference path through the center of the 3D laryngotracheal surface model for subsequent scaling operations. For example, the system may analyze the 3D laryngotracheal surface model to identify a central axis running through a laryngoscope positioned in a throat of the patient and continuing through the patient's airway, creating a path that follows the natural curvature of anatomical structures. The centerline computation enables more accurate measurements of internal diameters at various points along the airway.

At 904, method 900 includes computing a minimum inscribed sphere diameter along the centerline to calculate an inner diameter of the laryngoscope in the 3D surface model. Within a portion of the 3D laryngotracheal surface model where the laryngoscope is known to be positioned, at each point along the computed centerline, the system determines the largest sphere that can fit within the 3D laryngotracheal surface model without intersecting any walls. The diameter of this sphere represents the inner diameter of the structure at that specific location. This approach provides a mathematically robust method for measuring the internal dimensions of tubular structures like the laryngoscope and airway. For example, the system may create a series of virtual spheres centered at sequential points along the centerline, expanding each sphere until the sphere contacts an inner surface of the model, thereby determining a maximum possible diameter at each point.

At 906, method 900 includes identifying a minimum diameter of the laryngoscope. Among all the inscribed sphere diameters calculated along the portion of the centerline that passes through the laryngoscope, a smallest value is identified, a minimum diameter that represents a narrowest point of the laryngoscope in the 3D model. In various examples, the identification process may involve analyzing a plot of inscribed sphere diameters versus distance along the centerline, and locating a local minimum within the region corresponding to the laryngoscope, as shown in FIG. 5. For instance, the system might determine that the minimum internal diameter occurs at a specific distance from an entry point of the laryngoscope.

At 908, method 900 includes retrieving dimensions of the laryngoscope from a lookup table, based on the minimum internal diameter. The system may access a database or lookup table storing known physical dimensions of various laryngoscope models. By matching the relative proportions of the minimum internal diameter identified in the 3D model with the standardized dimensions in the lookup table, the system can identify the specific laryngoscope model used during the endoscopy. For example, if the system determines that the minimum internal diameter corresponds to a Parsons 3 laryngoscope, it retrieves the known physical dimension of 12 mm for this specific model from the lookup table.

At 910, method 900 includes determining a scaling ratio based on the retrieved laryngoscope dimensions. The system calculates a scaling factor by comparing the minimum internal diameter measured in the 3D model (in arbitrary units) with the known physical dimension of the corresponding laryngoscope (in millimeters), in accordance with equation 1 below:

$${f}_{scale} = \frac{{D}_{Real , Laryngoscope} ( mm )}{{D}_{SfM , Laryngascope ( )}} \qquad 1$$

The f_{scale} ratio establishes the relationship between the arbitrary units of the 3D reconstruction and real-world measurements. For example, if the minimum internal diameter in the 3D model is 80 arbitrary units, and the known physical dimension of the identified laryngoscope is 12 mm, the scaling ratio would be 12 mm/80 units=0.15 mm per unit.

At 912, method 900 includes scaling the 3D laryngotracheal surface model by the scaling ratio. The system applies the calculated scaling factor uniformly to 3D laryngotracheal surface model, transforming it from arbitrary units to real-world dimensions. This scaling operation ensures that all measurements derived from the model, such as airway diameters, stenosis lengths, or tissue thicknesses, accurately reflect the actual physical dimensions of the patient's anatomy. For example, once the scaling ratio is applied, clinicians can make precise measurements of a subglottic stenosis, determining its exact diameter and length in millimeters rather than arbitrary units. This transformation from relative to absolute measurements is directed at clinical decision-making, surgical planning, and quantitative assessment of airway pathologies.

In this way, method 900 provides a systematic approach to scaling the 3D surface model generated through SfM techniques to real-world dimensions. By leveraging the known physical dimensions of the laryngoscope used during the endoscopic procedure, the method establishes an accurate scaling reference that enables quantitative analysis of the patient's laryngotracheal anatomy.

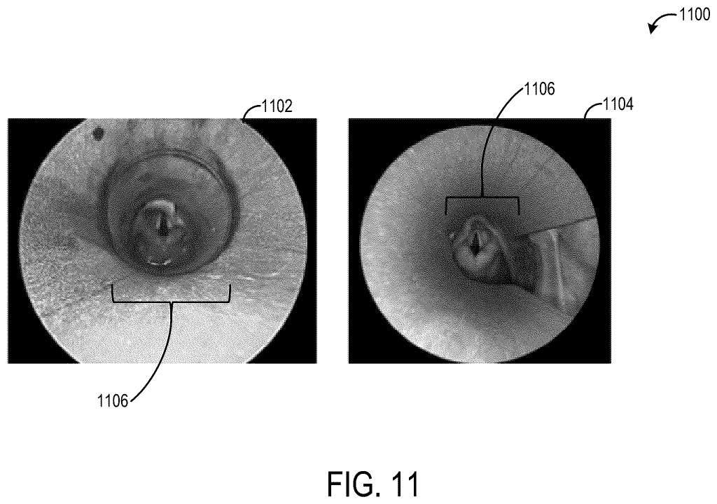

FIG. 11 shows example images 1102 and 1104 of a laryngoscope 1100 included in the contrast-enhanced endoscopic video sequence used in methods 600 and 900. An internal diameter 1106 of the laryngoscope 1100 is shown in both images. The laryngoscope 1100 is an appropriate object to use as a scaling reference because it satisfies basic criteria for method 900, namely, that it is rigid, has a surface roughness or defined features such as edges or points that enables scale-invariant feature detection, and is visually connected to the anatomy of interest in at least one single image. Additionally, the hollow tube form of the laryngoscope 1100 enables easy algorithmic measurements of the internal diameter 1106. During acquisition of the endoscopic video sequence, images of the laryngoscope 1100 are taken from sufficiently varied perspectives to facilitate accurate feature detection.

FIG. 12 illustrates an exemplary scenario 1200 where an anatomical region of a patient may be identified using measurements taken from a scaled 3D laryngotracheal surface model 1201 of the patient, as an alternative to identifying the anatomical region in an image of the patient acquired using an imaging modality such as CT or MRI that exposes the patient to radiation or requires general anesthesia. The scaled 3D laryngotracheal surface model 1201 may be a non-limiting example of the scaled 3D surface model 316 of FIG. 3, and may be generated by following method 600 of FIG. 6 and the other methods described herein. In FIG. 12, a glottis 1205 of the patient is identified from the measurements. In other examples, a different anatomical region of the patient may be identified from the measurements.

The measurements of the scaled 3D laryngotracheal surface model 1201 may be taken with respect to a centerline 1203 of the scaled 3D laryngotracheal surface model 1201 (e.g., centerline 503), which may be determined as described above in reference to FIG. 5. A measurement algorithm may be performed with respect to a portion 1202 of the scaled 3D laryngotracheal surface model 1201 including a trachea of the patient (e.g., trachea portion 317 of FIG. 3), in which the glottis is located. The algorithm may extract a plurality of cross-sectional planes 1204 at regular intervals (e.g., distances) along the centerline 1203 within the portion 1202 of the scaled 3D laryngotracheal surface model 1201, from a defined starting point of centerline 1203. A normal vector of each cross-sectional plane 1204 of the plurality of cross-sectional planes 1204 may be parallel to the centerline at a respective interval. As the centerline 1203 may not be perfectly straight, the cross-sectional planes 1204 may not be parallel to each other.

The algorithm may then determine bounds of each cross-sectional plane 1204 where a respective cross-sectional plane 1204 intersects with an inner surface 1208 of the scaled 3D laryngotracheal surface model 1201. Measurements of each bounded cross-sectional plane 1204 may then be taken or calculated, such as an area and a circular equivalent diameter. In one example, the circular equivalent diameter is calculated from the area using equation 2 below:

$${D}_{CE} = \sqrt{4 Area / π} \qquad 2$$

The circular equivalent diameter may then be used to determine a location of the glottis. For example, the circular equivalent diameters may be plotted against a distance along the centerline 1203, as shown by a line 1212 in plot 1210. A minimum circular equivalent diameter may be detected at a point 1214 of the line 1212, at a distance of 62 mm along the centerline from the defined starting point, which may provide the location of the glottis in both of the scaled 3D laryngotracheal surface model 1201 and the real anatomy of the patient. Various anatomical measurements can be further extracted from the circular-equivalent diameters of the bounded cross-sectional planes 1204 and cross-sectional area evolution curves, such as a length of a stenosis or narrowing, a ratio of a subglottic diameter to a diameter of the trachea, an aspect ratio through the subglottis, and/or other measurements. Figures such as the plot 1210 and tabulated metrics may be exported and shared as a report for clinicians and/or patients.

Thus, systems and methods are disclosed herein for using SfM techniques to reconstruct 3D surface models of internal patient anatomies, such as the larynx and trachea, from clinical endoscopy videos. While SfM techniques have been used to reconstruct outer surfaces of solid anatomical structures, applying them to internal surfaces is complicated by challenges including a sparsity of distinctive visual features on the surfaces, distortion resulting from varying optical parameters of endoscopic cameras, drift in image registration over time, and scaling surface models to real-world anatomical dimensions relied on for accurate anatomical measurements. The disclosed methods address these challenges by pre-processing endoscopic images using a calibration matrix to reduce distortion, increasing a contrast of scale-invariant features on the internal surfaces via a contrast enhancement algorithm, and in particular, by selecting an optimal starting image from which to begin the reconstruction process by ranking endoscopic images based on matched features in a sub-region of interest near the spatial center of the 3D model. The scaling problem is addressed by advantageously using a laryngoscope as a scaling reference, where dimensions of the laryngoscope are determined via a minimum-diameter calculation based on endoscopic image data, and the dimensions are used to scale a resulting 3D surface model to the patient anatomy. In this way, a cost-effective and radiation-free alternative to imaging studies is provided for quantitative analysis of complex laryngotracheal geometries including airway caliber and shape. The disclosed methods may improve understanding of upper airway disease including vocal fold paralysis, laryngeal cancer, subglottic hemangiomas, subglottic stenosis, tracheal stenosis, tracheal sleeves, and tracheomalacia. Results have demonstrated excellent resolution of reconstructions, when compared to high-resolution computed tomography (CT) scans (surface errors <0.300 mm).

The technical effect of providing 3D surface models of internal patient anatomies using SfM, enabled by the methods disclosed herein, is that accurate anatomical measurements of the anatomies may be facilitated without relying on imaging studies, which are more resource intensive to schedule, more computationally intensive to perform, and occupy a greater amount of memory resources to store. That is, by modeling the internal surface of the anatomies, and not modeling a full 3D volume of the anatomies, an amount of overall computation and memory use may be advantageously reduced.

The disclosure also provides support for a method, comprising: capturing a sequential motion picture of an internal three-dimensional (3D) surface of a patient anatomy using an endoscopic image capturing device, applying a contrast-enhancement algorithm to the captured sequential motion picture to create a contrast-enhanced endoscopic video sequence, reconstructing a 3D surface model of the patient anatomy from the contrast-enhanced endoscopic video sequence using structure from motion (SfM) photogrammetry, scaling the 3D surface model to real-world dimensions of the patient anatomy, to generate a scaled 3D surface model, calculating one or more measurements of the patient anatomy using the scaled 3D surface model, and displaying the one or more measurements on a display device and/or storing the one or more measurements in a memory. In a first example of the method, the endoscopic image capturing device comprises an endoscopic camera. In a second example of the method, optionally including the first example, the patient anatomy includes a larynx and a trachea of the patient, and the 3D surface model is a 3D laryngotracheal surface model. In a third example of the method, optionally including one or both of the first and second examples, the method further comprises: acquiring a plurality of images of a calibration object using the endoscopic camera, defining a calibration baseline matrix from the acquired images of the calibration object, the calibration baseline matrix establishing one or more optical parameters of the endoscopic camera, and reducing a distortion of one or more images of the contrast-enhanced endoscopic video sequence using the calibration baseline matrix. In a fourth example of the method, optionally including one or more or each of the first through third examples, the calibration object is a planar checkerboard. In a fifth example of the method, optionally including one or more or each of the first through fourth examples, the one or more optical parameters include a focal length of the endoscopic camera, a center of an image, and one or more distortion coefficients. In a sixth example of the method, optionally including one or more or each of the first through fifth examples, reconstructing the 3D surface model of the patient anatomy from the contrast-enhanced endoscopic video sequence using SIM photogrammetry further comprises: detecting one or more scale-invariant features in a plurality of images of the contrast-enhanced endoscopic video sequence, matching images of the plurality of images that include the same scale-invariant features, determining one or more poses of the endoscopic camera in the plurality of images, and matching each scale-invariant feature with a corresponding pose of the one or more poses of the endoscopic camera. In a seventh example of the method, optionally including one or more or each of the first through sixth examples, scaling the 3D surface model to the real-world dimensions of the patient anatomy to generate the scaled 3D surface model further comprises: acquiring an image of a target object using the endoscopic camera, the target object having known dimensions, a portion of the patient anatomy also visible in the image, determining a scaling ratio based on the known dimensions, and scaling the 3D surface model by the scaling ratio. In a eighth example of the method, optionally including one or more or each of the first through seventh examples, the target object is a laryngoscope inserted into an airway of the patient, and determining the scaling ratio based on the known dimensions further comprises: computing a centerline of an airway of the 3D surface model in a portion of the 3D surface model including the laryngoscope, computing an inner diameter of the laryngoscope by computing a minimum sphere diameter along the centerline that can be inscribed in the 3D surface model, retrieving dimensions of the laryngoscope from a lookup table stored in the memory, based on the inner diameter, and determining the scaling ratio based on the retrieved laryngoscope dimensions. In a ninth example of the method, optionally including one or more or each of the first through eighth examples, determining the one or more poses of the endoscopic camera in the plurality of images and matching each scale-invariant feature with the corresponding pose of the one or more poses of the endoscopic camera further comprises: identifying a first image of the contrast-enhanced endoscopic video sequence acquired as a tip of an endoscope of the endoscopic camera first passes through an anatomical region of interest of the patient, selecting a plurality of images of the contrast-enhanced endoscopic video sequence subsequent to the first image, ranking each image of the plurality of subsequent images based on a number of matching scale-invariant features in the image, selecting an image of the plurality of subsequent images having a highest rank as a starting image, and determining the one or more poses of the endoscopic camera in the plurality of images and matching each scale-invariant feature with the corresponding pose of the one or more poses of the endoscopic camera starting at the starting image. In a tenth example of the method, optionally including one or more or each of the first through ninth examples, the 3D surface model is a 3D laryngotracheal surface model and the anatomical region of interest includes vocal cords of the patient. In a eleventh example of the method, optionally including one or more or each of the first through tenth examples, calculating the one or more measurements of the patient anatomy using the scaled 3D surface model further comprises: computing a centerline of an airway of the scaled 3D surface model, extracting a plurality of cross-sectional planes at regular intervals along the centerline, a normal vector of each cross-sectional plane of the plurality of cross-sectional planes parallel to the centerline at a respective interval, determining bounds of each cross-sectional plane where a respective cross-sectional plane intersects with an inner surface of the scaled 3D surface model, calculating a circular equivalent diameter of each bounded cross-sectional plane based on the circular equivalent diameters of the bounded cross-sectional planes, extracting a measurement of an anatomical feature of the patient.

The disclosure also provides support for a system for reconstructing a model of an inner three-dimensional (3D) surface of a patient anatomy, the system comprising: an endoscope including an endoscopic camera, one or more processors, and a memory that stores executable instructions that, when executed, cause the one or more processors to: capture a sequential motion picture of the inner 3D surface using the endoscopic camera, apply a contrast-enhancement algorithm to the captured sequential motion picture to create a contrast-enhanced endoscopic video sequence, reconstruct a 3D surface model of the patient anatomy from the contrast-enhanced endoscopic video sequence using structure from motion (SfM) photogrammetry, scale the 3D surface model to real-world dimensions of the patient anatomy, to generate a scaled 3D surface model, calculate one or more measurements of the patient anatomy using the scaled 3D surface model, and display the one or more measurements on a display device and/or store the one or more measurements in a memory. In a first example of the system, the endoscope is a rigid endoscope. In a second example of the system, optionally including the first example, the inner 3D surface is a laryngotracheal surface of the patient, and further instructions are stored in the memory that when executed, cause the one or more processors to: acquire a plurality of images of a calibration object using the endoscopic camera, define a calibration baseline matrix from the acquired images of the calibration object, the calibration baseline matrix establishing one or more optical parameters of the endoscopic camera, and apply the calibration baseline matrix to one or more images of the contrast-enhanced endoscopic video sequence to reduce a distortion of the one or more images. In a third example of the system, optionally including one or both of the first and second examples, further instructions are stored in the memory that when executed, cause the one or more processors to: detect one or more scale-invariant features in a plurality of images of the contrast-enhanced endoscopic video sequence, match images of the plurality of images that include the same scale-invariant features, determine one or more poses of the endoscopic camera in the plurality of images, and triangulate each scale-invariant feature from a corresponding pose of the one or more poses of the endoscopic camera using a baseline calibration matrix. In a fourth example of the system, optionally including one or more or each of the first through third examples, further instructions are stored in the memory that when executed, cause the one or more processors to: acquire an image of a target object using the endoscopic camera, the target object having known dimensions, a portion of the patient anatomy also visible in the image, determine a scaling ratio based on the known dimensions, and scale the 3D surface model by the scaling ratio. In a fifth example of the system, optionally including one or more or each of the first through fourth examples, the target object is a laryngoscope inserted into an airway of the patient, and further instructions are stored in the memory that when executed, cause the one or more processors to: compute a centerline of an airway of the 3D surface model in a portion of the 3D surface model including the laryngoscope, compute an inner diameter of the laryngoscope by computing a minimum sphere diameter along the centerline that can be inscribed in the 3D surface model, retrieve dimensions of the laryngoscope from a lookup table stored in the memory, based on the inner diameter, and determine the scaling ratio based on the retrieved laryngoscope dimensions. In a sixth example of the system, optionally including one or more or each of the first through fifth examples, further instructions are stored in the memory that when executed, cause the one or more processors to: identify a first image of the contrast-enhanced endoscopic video sequence acquired as a tip of the endoscope first passes through vocal cords of the patient, select a plurality of images of the contrast-enhanced endoscopic video sequence subsequent to the first image, rank each image of the plurality of subsequent images based on a number of matching scale-invariant features in the image, select an image of the plurality of subsequent images having a highest rank as a starting image, and reconstruct the 3D surface model starting at the starting image.

The disclosure also provides support for a method, comprising: capturing an endoscopic video sequence of a three-dimensional (3D) laryngotracheal surface of a patient using an endoscopic camera inserted into a laryngoscope, enhancing a contrast of the endoscopic video sequence using a contrast-enhancement algorithm, reducing a distortion of the contrast-enhanced endoscopic video sequence using a baseline calibration matrix calculated using images of a calibration object acquired via the endoscopic camera, reconstructing a 3D surface model of the laryngotracheal surface from the contrast-enhanced endoscopic video sequence using structure from motion (SfM) photogrammetry, starting at a starting image, the starting image having a highest number of scale-invariant features included in other images of the contrast-enhanced endoscopic video sequence, computing an inner diameter of the laryngoscope by computing a minimum diameter of a sphere inscribed in the 3D surface model with a center on a centerline of an airway defined by the laryngotracheal surface, retrieving dimensions of the laryngoscope from a lookup table, based on the inner diameter, determining a scaling ratio based on the retrieved laryngoscope dimensions, scaling the 3D surface model by the scaling ratio to generate a scaled 3D laryngotracheal surface model having a same scale as the laryngotracheal surface of the patient, calculating one or more measurements of the laryngotracheal surface of the patient using the scaled 3D surface model, and displaying the one or more measurements on a display device and/or storing the one or more measurements in a memory.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and it is not a requirement that all embodiments exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

1. A method, comprising:
capturing a sequential motion picture of an internal three-dimensional (3D) surface of a patient anatomy using an endoscopic image capturing device;
applying a contrast-enhancement algorithm to the captured sequential motion picture to create a contrast-enhanced endoscopic video sequence;
reconstructing a 3D surface model of the patient anatomy from the contrast-enhanced endoscopic video sequence using structure from motion (SfM) photogrammetry;
scaling the 3D surface model to real-world dimensions of the patient anatomy, to generate a scaled 3D surface model;
calculating one or more measurements of the patient anatomy using the scaled 3D surface model; and
displaying the one or more measurements on a display device and/or storing the one or more measurements in a memory.

2. The method of claim 1, wherein the endoscopic image capturing device comprises an endoscopic camera.

3. The method of claim 2, wherein the patient anatomy includes a larynx and a trachea of the patient, and the 3D surface model is a 3D laryngotracheal surface model.

4. The method of claim 2, further comprising:
acquiring a plurality of images of a calibration object using the endoscopic camera;
defining a calibration baseline matrix from the acquired images of the calibration object, the calibration baseline matrix establishing one or more optical parameters of the endoscopic camera; and
reducing a distortion of one or more images of the contrast-enhanced endoscopic video sequence using the calibration baseline matrix.

5. The method of claim 4, wherein the calibration object is a planar checkerboard.

6. The method of claim 4, wherein the one or more optical parameters include a focal length of the endoscopic camera, a center of an image, and one or more distortion coefficients.

7. The method of claim 3, wherein reconstructing the 3D surface model of the patient anatomy from the contrast-enhanced endoscopic video sequence using SfM photogrammetry further comprises:
detecting one or more scale-invariant features in a plurality of images of the contrast-enhanced endoscopic video sequence;
matching images of the plurality of images that include the same scale-invariant features;
determining one or more poses of the endoscopic camera in the plurality of images; and
matching each scale-invariant feature with a corresponding pose of the one or more poses of the endoscopic camera.

8. The method of claim 3, wherein scaling the 3D surface model to the real-world dimensions of the patient anatomy to generate the scaled 3D surface model further comprises:
acquiring an image of a target object using the endoscopic camera, the target object having known dimensions, a portion of the patient anatomy also visible in the image;
determining a scaling ratio based on the known dimensions; and
scaling the 3D surface model by the scaling ratio.

9. The method of claim 8, wherein the target object is a laryngoscope inserted into an airway of the patient, and determining the scaling ratio based on the known dimensions further comprises:
computing a centerline of an airway of the 3D surface model in a portion of the 3D surface model including the laryngoscope;
computing an inner diameter of the laryngoscope by computing a minimum sphere diameter along the centerline that can be inscribed in the 3D surface model;
retrieving dimensions of the laryngoscope from a lookup table stored in the memory, based on the inner diameter; and
determining the scaling ratio based on the retrieved laryngoscope dimensions.

10. The method of claim 7, wherein determining the one or more poses of the endoscopic camera in the plurality of images and matching each scale-invariant feature with the corresponding pose of the one or more poses of the endoscopic camera further comprises:
identifying a first image of the contrast-enhanced endoscopic video sequence acquired as a tip of an endoscope of the endoscopic camera first passes through an anatomical region of interest of the patient;
selecting a plurality of images of the contrast-enhanced endoscopic video sequence subsequent to the first image;
ranking each image of the plurality of subsequent images based on a number of matching scale-invariant features in the image;
selecting an image of the plurality of subsequent images having a highest rank as a starting image; and
determining the one or more poses of the endoscopic camera in the plurality of images and matching each scale-invariant feature with the corresponding pose of the one or more poses of the endoscopic camera starting at the starting image.

11. The method of claim 10, wherein the 3D surface model is a 3D laryngotracheal surface model and the anatomical region of interest includes vocal cords of the patient.

12. The method of claim 1, wherein calculating the one or more measurements of the patient anatomy using the scaled 3D surface model further comprises:
computing a centerline of an airway of the scaled 3D surface model;
extracting a plurality of cross-sectional planes at regular intervals along the centerline, a normal vector of each cross-sectional plane of the plurality of cross-sectional planes parallel to the centerline at a respective interval;
determining bounds of each cross-sectional plane where a respective cross-sectional plane intersects with an inner surface of the scaled 3D surface model;
calculating a circular equivalent diameter of each bounded cross-sectional plane based on the circular equivalent diameters of the bounded cross-sectional planes, extracting a measurement of an anatomical feature of the patient.

13. A system for reconstructing a model of an inner three-dimensional (3D) surface of a patient anatomy, the system comprising:
an endoscope including an endoscopic camera;
one or more processors, and a memory that stores executable instructions that, when executed, cause the one or more processors to:
capture a sequential motion picture of the inner 3D surface using the endoscopic camera;
apply a contrast-enhancement algorithm to the captured sequential motion picture to create a contrast-enhanced endoscopic video sequence;
reconstruct a 3D surface model of the patient anatomy from the contrast-enhanced endoscopic video sequence using structure from motion (SfM) photogrammetry;
scale the 3D surface model to real-world dimensions of the patient anatomy, to generate a scaled 3D surface model;
calculate one or more measurements of the patient anatomy using the scaled 3D surface model; and
display the one or more measurements on a display device and/or store the one or more measurements in a memory.

14. The system of claim 13, wherein the endoscope is a rigid endoscope.

15. The system of claim 13, wherein the inner 3D surface is a laryngotracheal surface of the patient, and further instructions are stored in the memory that when executed, cause the one or more processors to:
acquire a plurality of images of a calibration object using the endoscopic camera;
define a calibration baseline matrix from the acquired images of the calibration object, the calibration baseline matrix establishing one or more optical parameters of the endoscopic camera; and
apply the calibration baseline matrix to one or more images of the contrast-enhanced endoscopic video sequence to reduce a distortion of the one or more images.

16. The system of claim 13, wherein further instructions are stored in the memory that when executed, cause the one or more processors to:
detect one or more scale-invariant features in a plurality of images of the contrast-enhanced endoscopic video sequence;
match images of the plurality of images that include the same scale-invariant features;
determine one or more poses of the endoscopic camera in the plurality of images; and
triangulate each scale-invariant feature from a corresponding pose of the one or more poses of the endoscopic camera using a baseline calibration matrix.

17. The system of claim 13, wherein further instructions are stored in the memory that when executed, cause the one or more processors to:
acquire an image of a target object using the endoscopic camera, the target object having known dimensions, a portion of the patient anatomy also visible in the image;
determine a scaling ratio based on the known dimensions; and
scale the 3D surface model by the scaling ratio.

18. The system of claim 17, wherein the target object is a laryngoscope inserted into an airway of the patient, and further instructions are stored in the memory that when executed, cause the one or more processors to:
compute a centerline of an airway of the 3D surface model in a portion of the 3D surface model including the laryngoscope;
compute an inner diameter of the laryngoscope by computing a minimum sphere diameter along the centerline that can be inscribed in the 3D surface model;
retrieve dimensions of the laryngoscope from a lookup table stored in the memory, based on the inner diameter; and
determine the scaling ratio based on the retrieved laryngoscope dimensions.

19. The system of claim 13, wherein further instructions are stored in the memory that when executed, cause the one or more processors to:
identify a first image of the contrast-enhanced endoscopic video sequence acquired as a tip of the endoscope first passes through vocal cords of the patient;
select a plurality of images of the contrast-enhanced endoscopic video sequence subsequent to the first image;
rank each image of the plurality of subsequent images based on a number of matching scale-invariant features in the image;
select an image of the plurality of subsequent images having a highest rank as a starting image; and
reconstruct the 3D surface model starting at the starting image.

20. A method, comprising:
capturing an endoscopic video sequence of a three-dimensional (3D) laryngotracheal surface of a patient using an endoscopic camera inserted into a laryngoscope;
enhancing a contrast of the endoscopic video sequence using a contrast-enhancement algorithm;
reducing a distortion of the contrast-enhanced endoscopic video sequence using a baseline calibration matrix calculated using images of a calibration object acquired via the endoscopic camera;
reconstructing a 3D surface model of the laryngotracheal surface from the contrast-enhanced endoscopic video sequence using structure from motion (SfM) photogrammetry, starting at a starting image, the starting image having a highest number of scale-invariant features included in other images of the contrast-enhanced endoscopic video sequence;
computing an inner diameter of the laryngoscope by computing a minimum diameter of a sphere inscribed in the 3D surface model with a center on a centerline of an airway defined by the laryngotracheal surface;
retrieving dimensions of the laryngoscope from a lookup table, based on the inner diameter;
determining a scaling ratio based on the retrieved laryngoscope dimensions;
scaling the 3D surface model by the scaling ratio to generate a scaled 3D laryngotracheal surface model having a same scale as the laryngotracheal surface of the patient;
calculating one or more measurements of the laryngotracheal surface of the patient using the scaled 3D surface model; and
displaying the one or more measurements on a display device and/or storing the one or more measurements in a memory.
